(19) United States
(12) Patent Application Publication
(10) Pub. No.: US 2010/0233793 A1
(43) Pub. Date: Sep. 16, 2010

(54) HEALTHCARE MANAGEMENT APPARATUS, HEALTHCARE MANAGEMENT METHOD, AND DISPLAY METHOD OF DETERMINATION RESULTS

(75) Inventors: Ai TSUTSUI, Osaka (JP); Junko Mikata, Osaka (JP); Seung-jin Cho, Osaka (JP)

Correspondence Address: BIRCH STEWART KOLASCH & BIRCH, PO BOX 747, FALLS CHURCH, VA, 22040-0747, US

(73) Assignee: SHARP KABUSHIKI KAISHA, Osaka-shi (JP)

(21) Appl. No.: 12/723,304

(22) Filed: Mar. 12, 2010

(30) Foreign Application Priority Data
Mar. 13, 2009 (JP) 2009-061777

(51) Int. Cl. C12M 1/00; G06Q 50/00

(52) U.S. Cl. 435/287.1; 705/2

(57) ABSTRACT

A healthcare management apparatus for determining a health condition of subjects is provided.

The following configuration realizes the above object.

The healthcare management apparatus includes an acquiring means for acquiring physical data and blood components data of subjects, data conversion formula and data determination criteria; and a processing means for converting the physical data and the blood components data into relative values by using the data conversion formula, and for determining health condition of the subjects by using the data determination criteria.

Preferably, the acquiring means further comprise health assessment formula and health condition determination criteria. The processing means calculates a health condition determination relative values by combining two or more of the relative values and/or the health condition determination relative values and using total health assessment formula and determines a total health condition of subjects by checking the total health condition determination relative values with total health condition determination criteria. The acquiring means also includes a memory and a communication means.

### BACKGROUND OF THE INVENTION

1. Field of the Invention

The present invention relates to an apparatus and a method for proposing the healthcare management using physical data and blood components data and thus determining the health conditions of the subjects.

2. Background Art

In recent years, many people have been suffering from cardiovascular diseases due to lifestyle diseases such as diabetes and high blood pressure. Lifestyle diseases, which sometimes occur resulting from genetic factors, can be generally prevented by improvement of daily life such as diet and exercise. In this circumstance, National Cholesterol Education Program (NCEP) has published a simple definition that determines as metabolic syndrome when any three of five diagnostic items (waist circumference, blood glucose level, blood pressure, neutral fat level and HDL cholesterol level) meet its criteria.

Therefore, it is required to build a system to easily manage the risk for progression to lifestyle diseases and to allow subjects to improve lifestyle in the early stage.

To determine the lifestyle diseases, it is necessary to measure the amount of the substances (glucose, proteins, lipids, etc.) contained in biological samples such as blood. In addition, in order to understand the risks for progression of lifestyle diseases, biological samples such as saliva, blood and urine may be used in determination of health condition. However, since the amounts of the substances contained in the biological samples are greatly differed between individuals, there is a problem of determining health condition or medical conditions only by the measurement results of substances.

Patent documents 1 to 4 suggest a method for determining health condition of subjects using biological samples. Patent document 5 suggests an analytical microchip sensor that could apply to measurement of substances in biological samples.

It is reported in Non-patent documents 1 to 6 that the relationship between blood components (proteins) and the risk of lifestyle diseases.

### PRIOR ART DOCUMENTS

### Patent Documents

Patent document 1: JP 2008-23199
Patent document 2: JP 2007-33410
Patent document 3: JP 2007-275287
Patent document 4: JP 2002-14095
Patent document 5: JP 2008-203158

### Non-Patent Documents

Non-patent document 1
“Adiponectin and the receptors thereof”, Fuji medical publishing

Non-patent document 2
Satoh N et al, Leptin-to-adiponectin ratio as a potential atherogenic index in a obese type 2 diabetic patients, DIABETES CARE, vol 27, 2488-2490, 2004

Non-patent document 3
Arita Y et al, Paradoxical decrease of an adipose-specific protein adiponectin, in obesity, Biochem Biophys Res Commun. 257, 79-83, 1999

Non-patent document 4
Hotta K et al, plasma level of a novel adipose-specific protein, adiponectin, in type 2 diabetic patients, Arterioscler Thromb Basc Biol 20; 1595-1599, 2000

Non-patent document 5
On Y-K et al, Serum resistin as a biological marker for coronary artery disease and restenosis in type 2 diabetic patients, Circ. J. 71, 868-873, 2007

Non-patent document 6
S. Gwozdziewiczova et al, TNF-α in the development of insulin resistance and other disorders in metabolic syndrome, Biomed. Papers 149(1), 109-117, 2005

Patent document 1 proposes an apparatus that can determine whether a subject to be metabolic syndrome or not, by inputting parameters as the criteria of metabolic syndrome (waist circumference, blood pressure, plasma glucose level, HDL cholesterol level, neutral fat level) with the sexes separated.

However, this technology provides only the determination of metabolic syndrome according to standards of Japan Society for the Study of Obesity (JASSO). Therefore, it is difficult to precisely determine the progression to lifestyle diseases due to other reasons except metabolic syndrome.

Patent document 2 proposes a method to determine the metabolic syndrome that is closely related to lifestyle disease with measuring the choline-type plasmalogen and ethanol-amine-type plasmalogen level in serum.

However, this technology shows only low correlation between the measured plasmalogen level in serum and lifestyle diseases (diabetes, arteriosclerosis, hypertension, hyperlipidemia, etc.), therefore, it is difficult to exactly determine metabolic syndrome and the risk of lifestyle diseases.

Patent document 3 proposes a method to determine the degree of stress by using a combination of salivary ingredients and indicators other than salivary components.

However, in this technology, the diagnosis of stress shows only “Normal” or “Attention” and the determination range of each area is wide. Therefore, there is a problem of being difficult for users to understand the degree of improvement in symptoms.

Patent document 4 proposes a method to collect data of several components in blood and then to make a diagnosis using a pattern of such measurements.

In this technique, a diagnosis is made using several items of blood components as indices. However, because this technology only provides a list of the measurements (absolute values) and determines whether the values are normal or not, the user do not exactly understand the risk. Moreover, although several items of blood components are used as indices, the relation among the items is not identified clearly.

The relation between blood components and lifestyle diseases described in Non-patent documents 1 to 6 is as follows.

### (Adiponectin)

It is known that adiponectin level in blood is decreased with obesity and insulin resistance while the level is increased because of body weight loss (improvements in obesity). In addition, the previous research has revealed anti-diabetic and anti-atherogenic effects of adiponectin (cf. Non-patent documents 1, 3 and 4.).

### (Leptin)

Leptin is produced in fat cells, and conveys the amount of body fat to the brain to adjust metabolism and appetite, thus having the function of appetite control (cf. Non-patent document 2.).

### (Resistin)

Resistin is One Kind of Adipokine, which Causes Insulin Resistance. Resistin causes insulin resistance at fat cells, muscle cells, liver cells and the like, and thus is said to be the cause of increasing glucose in blood. In addition, resistin increases the production of endothelin that is said to be the cause of high blood pressure, thus may promote arterial sclerosis, and is deeply involved in lifestyle diseases including metabolic syndrome (cf. Non-patent literature 5.).

### (TNF-α)

The secretion amount of TNF-α is increased when fat cells enlarge. TNF-α serves to inhibit the activation of insulin receptors in fat cells and muscle cells, and thus may lead to insulin resistance and high concentration of glucose in blood. Therefore, TNF-α may cause a type 2 diabetes (cf. Non-patent document 6.).

### SUMMARY OF THE INVENTION

It is difficult to accurately grasp a health condition only by measuring substances that serves as indices of the health condition, and stress-induced diseases and by comparing them with the criteria value of each substance. Therefore, a health condition management apparatus is required that can easily determine the health condition of subjects accurately and the risk of progression to lifestyle diseases.

Conventional apparatuses for determining metabolic syndrome, which use the definition of National Cholesterol Education Program (NCEP), the International Diabetes Federation (IDF), Japan Society for the Study of Obesity (JASSO) or the like, can easily make the determination of metabolic syndrome. However, only by the determination of metabolic syndrome, it is difficult to predict the possibility or risk of progression to lifestyle diseases in view of health condition of subjects.

The present invention is made in cultivating of the problems associated with the conventional technologies. The object of the present invention is to provide an apparatus and a method for determining health condition, which can precisely determine personal health condition and the risk of progression to lifestyle diseases and thus allows a proposal of improvement in health to the people. Moreover, the present invention aims to provide an apparatus and a method for displaying the health condition results so that the subjects may visually understand their health condition, which results in early improvement in health.

The present invention relating to a health condition determination apparatus to solve the above problems is characterized by comprising:

an acquiring means for obtaining physical data and blood components data of subjects, data conversion formula and data determination criteria; and

a processing means for converting the physical data and the blood components data into relative values by using the data conversion formula, and for determining health condition of the subjects by using the criteria for determining data.

With this apparatus, the health condition of the subjects and the risk of progression to lifestyle diseases can be easily and accurately determined. In addition, the improvement in lifestyle can be proposed to the subjects according to the results of the health condition determination, and thus the progression to lifestyle diseases can be prevented in advance.

Herein, the physical data include data from a subject's body appearance that can be easily measured, such as body height, body weight, BMI (Body mass index), blood pressure, body temperature, heart rate, respiration rate and body fat percentage. Also, blood components data include the data analyzed using a blood sample such as blood glucose level, cholesterol level, neutral fat level, white blood cell count, red blood cell count, hematocrit level and various proteins level.

In the above configuration, the acquiring means may further comprise health assessment formula and health condition determination criteria, and the processing means combines two or more of the physical data, the blood components data and the above relative values to calculate health condition determination relative values using the health assessment formula, and check the health condition determination relative values with the health condition determination criteria to determine the risk of progression to lifestyle diseases based on health condition of subjects.

It is preferable to combine several physical data and blood components data in determination for the risk of progression to lifestyle diseases such as metabolic syndromes, hyperlipidemia and high blood pressure. However, some measured data itself cannot be simply combined because of significant differences in units or numbers itself. On the other hand, since relative values of the measured values is easy to be combined, the health condition and the risk of progression to lifestyle diseases can be easily determined.

Herein, the phrase “combines two or more of the physical data, the blood components data and the relative values” includes the case in which two or more elements are selected from one of the above three categories (the physical data, the blood components data and the relative values), the case in which one or more elements are respectively selected from two of the above three categories, and the case in which one or more elements are selected from each of the above three categories.

In the above configuration, the acquiring means may further comprise total health assessment formula and total health condition determination criteria, and the processing means combines two or more of the relative values and the health condition determination relative values to calculate total health condition determination relative values using the total health assessment formula, and then may check the total health condition determination relative values with the total health condition determination criteria to determine total health condition of subjects.

“In order to totally determine health condition, it is preferable to further combine two or more individual data and determination of the risk of progression to lifestyle diseases by understanding the health condition based on combination of a plurality of individual data. In this case, the combination of relative values is also useful.

As a data conversion formula, a pattern table showing the relationship between the relative values and the measurement values can be used.

In the above configuration, several kinds of pattern tables are obtained. The processing means determines at least one of sex, age and race of the subjects and then decides the pattern table to be used.

Some physical data and blood components data may significantly vary depending on the subject's sex, age and race. Therefore, use of the pattern table suitable for the subject can provide more precise determination of health condition. Moreover, other factors (being pregnant or not, an athlete or not, etc.) may be further determined, and then another pattern table may be decided in view of the factors.

In the above configuration, the health condition determination apparatus may further comprise an input means.

In the above configuration, the health condition determination apparatus may further comprise a display that informs the results of the determination.

In the above configuration, the acquiring means may further acquire health improvement data, and the processing means may check the determination results with the health improvement data and then may let the display show the determination results and the health improvement method corresponding thereto.

The display shows the determination results and the health improvement method corresponding thereto, which urges the subjects to improve their own health.

In the above configuration, the acquiring means may further acquire time course information on the determination results, and the display may further inform the monitoring of the results with time.

Displaying the time course information results in easily understanding whether the subject's health condition is being improved or not.

In the above configuration, the health condition determination apparatus further comprises detection instruments for analyzing blood components. The processing means calculates the blood components data from signals detected by the detection instruments.

The detection instruments can shorten the time from the analysis to the determination.

The acquiring means may comprise a memory for recording various information, such as a hard disk and a Flash SSD (Flash Solid State Drive), or a communication means for obtaining various information via electric communication lines such as Internet and other wired or wireless communication lines.

The first aspect of the present invention relating to a health condition determination method for solving the above problems is characterized by comprising:

a relative values calculating step for converting physical data and/or blood component data of subjects into relative values using data conversion formula; and

a data determination step for determine whether the relative values meet data determination criteria or not.

The above method allows the subjects to easily determine whether each data is in the High Risk range or not.

The above configuration may further comprise:

a health condition determination relative values calculating step for calculating relative values used in health condition determination by combining two or more of the relative values and using health assessment formula; and

a health condition determination step for determining the risk of progression to lifestyle diseases of the subjects by check the health condition determination relative values with health condition determination criteria.

According to this configuration, it becomes easy to combine the several data by using the relative values, and thus the health condition and the risk of progression to lifestyle diseases can be easily determined.

The health condition determination relative values calculation and the determination of the risk of progression to lifestyle diseases may be performed at the same time. In this case, the health assessment formula may be integrated with the health condition determination criteria.

The second aspect of the present invention relating to a health condition determination method for solving the above problems is characterized by comprising:

a data determination step for determining whether physical data and/or blood component data of subjects satisfy the data determination criteria or not;

a relative values conversion step for converting the physical data and the blood components data into relative values based on results of the data determination;

a health condition determination relative values calculating step for combining two or more of the relative values and then calculating relative values used in health condition determination by using health assessment formula; and

a health condition determination step for determining the risk of progression to lifestyle diseases of the subjects by check the health condition determination relative values with health condition determination criteria.

This configuration is similar to the above configuration except that each data is determined before the conversion into the relative value and then the data is converted into relative values on the basis of the determination. Even in this configuration, several data can be easily combined and thus the health condition can be easily determined.

The configuration may be also used in which the conversion of the data into the relative values is performed in conjunction with the data determination.

The above configuration of the first or second aspect of the present invention may further comprise,

a total health condition determination relative values calculating step for calculating relative values used in determination of total health condition of subjects, by combining two or more of the relative values and/or the health condition determination relative values and using total health assessment formula; and

a total health condition determination step for determining total health condition of the subjects by check the total health condition determination relative values with total health condition determination criteria.

In order to totally determine the health condition, it is preferable to further combine individual data or health condition determination based on combination of several individual data. Also in this case, a combination of relative values is useful.

In the above configuration, the total health condition relative values calculating and the total health condition determination may be performed at the same time. In this case, the total health assessment formula may be integrated with the total health condition determination criteria.

In the above configuration, the health condition determination relative values and/or the relative values may be at least one selected from the group consisting of the Body Mass Index relative values, the metabolic syndrome determination relative values and the specific proteins in blood determination relative values.

In order to determine the risk of progression to lifestyle diseases, it is preferable to determine the Body Mass Index relative values, the metabolic syndrome determination relative values and the specific proteins in blood determination relative values. Preferably, two or more kinds of these relative values are combined to determine. More preferably, all three kinds of these relative values are combined to determine. Thereby, metabolic syndrome and the risk of progression to lifestyle diseases can be determined in more detail.

The determination of metabolic syndrome may use the definition of National Cholesterol Education Program (NCEP), International Diabetes Federation (IDF) and the Japan Society of Obesity (JASSO). Or, other definitions also may be used.

In the specific proteins in blood determination, it is preferable to use the level in blood of at least one of adiponectin, leptin, resistin and TNF-α, but also other components in blood could be used.

As a data conversion formula, a pattern table showing the relationship between the measurement values and relative values can be used.

As a health assessment formula, a formula to sum up relative values in equal ratio or in different ratios for each data may be used. In addition, when one kind of relative values exceeds a criteria value, the health assessment relative values may be determined as “High Risk”, regardless of other relative values.

As a total health assessment formula, a formula to sum up relative values in equal ratio or in different ratios for each data may be used. In addition, when one kind of relative values or the total health condition relative values exceeds a criteria value, the total health assessment relative values may be determined as “High Risk”, regardless of other relative values.

The above configuration may further comprise a pattern table deciding step to determine at least one of sex, age, race of subjects and thus to decide the pattern table to be used.

The display method for showing the determination results according to the present invention is characterized by that the relative values of the analysis data on adiponectin, leptin, resistin and TNF-α are displayed together with graphs.

The expression of adiponectin, leptin, resistin and TNF-α in the relative values allows the risk to be easily understood.

The display form according to the present invention may show only the relative values, or the absolute values together with the corresponding relative value.

In the above configuration, the graph is preferably arranged so that indices that have a strong degree of the antagonistic relationship and/or correlation are placed adjacently to each other.

The above configuration may comprise a system in which time course information on the measurement values of the above-mentioned four components is shown together with graphs.

Displaying the time course information facilitates to understand whether the health condition of the subjects is getting better or not. The time course information may be displayed all times or only when commanded to display it.

In the above configuration, when any of the determination results of the above four components exceeds to the criteria value, a warning message may be displayed.

With the warning message, the risk of progression to lifestyle disease of subjects can be more easily understood.

In the above configuration, a health improvement method of the subjects may be displayed together with the above determination results.

In the above configuration, when X1 is an adiponectin level, X2 is a leptin level, X3 is a resistin level, and X4 is a TNF-α level, and when coefficients of obesity degree determined by Body Mass Index relative values are defined as Y1 and Y2, X1Y1, X2Y2, X3Y2 and X4Y1 may be displayed.

In the above configuration, the parameters may be shown using a human-shaped model, which displays X1Y1 at the right edge of the waist, X2Y2 at the right edge of the chest, X3Y2 at the left edge of the chest, and X4Y1 at the left edge of the waist. For this display, each of the levels and values is multiplied by coefficients so as to be displayed at the appropriate position.

In the above configuration, the parameters may be shown using another human-shaped model, which displays X1Y1 at the back edge of the waist, X2Y2 at the back edge of the abdomen, X3Y2 at the front edge of the abdomen, and X4Y1 at the front edge of the waist.

The use of such display forms facilitates to understand health condition visually.

In the present invention, in order to determine health condition of the subjects, the physical data and blood data are converted into the relative values. The combination of the relative values of the several data provides the exact determination of health condition.

Also, displaying the determination results as well as a health improvement method according to subjects' health condition allows the improvement and prevention of lifestyle diseases, and thus facilitates health control of the subjects.

In addition, since the health condition of the subjects is visually and clearly displayed, effects of the improvement and prevention of lifestyle diseases can be enhanced.

### BRIEF DESCRIPTION OF THE DRAWINGS

FIG. 1 is a block diagram showing the configuration of the healthcare management apparatus according to the present invention.

FIG. 2 is a block diagram showing the configuration of the processing means used in the healthcare management apparatus according to the present invention.

FIG. 3 is a diagram showing the display method for showing the results of the total health condition determination according to Embodiment 1.

FIG. 4 is a diagram showing a flow chart of the health condition determining method according to Embodiment 1.

FIG. 5 is a diagram showing a flow chart of the health condition determining method according to Embodiment 2.

FIG. 6 is a diagram showing a flow chart of the determining method for metabolic syndrome according to Embodiment 2.

FIG. 7 is a diagram showing a flow chart of the determining method for BMI according to Embodiment 2.

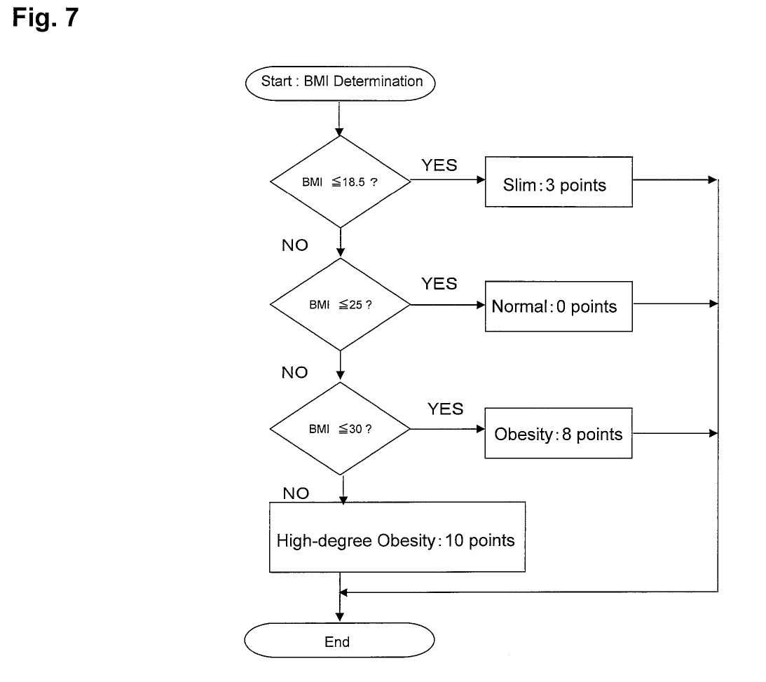

FIG. 8 is a diagram showing a flow chart of the health condition determining method according to Embodiment 2.

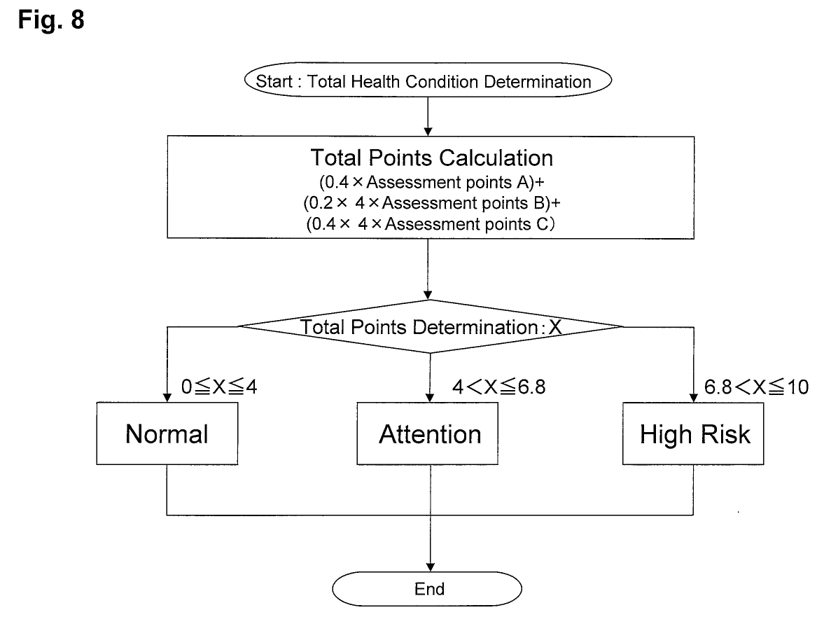

FIG. 9 is a diagram showing a display form of the determination results according to Embodiment 4.

FIG. 10 is a diagram showing another display form of the determination results according to Embodiment 4.

### DETAILED DESCRIPTION OF THE INVENTION

### Embodiment 1

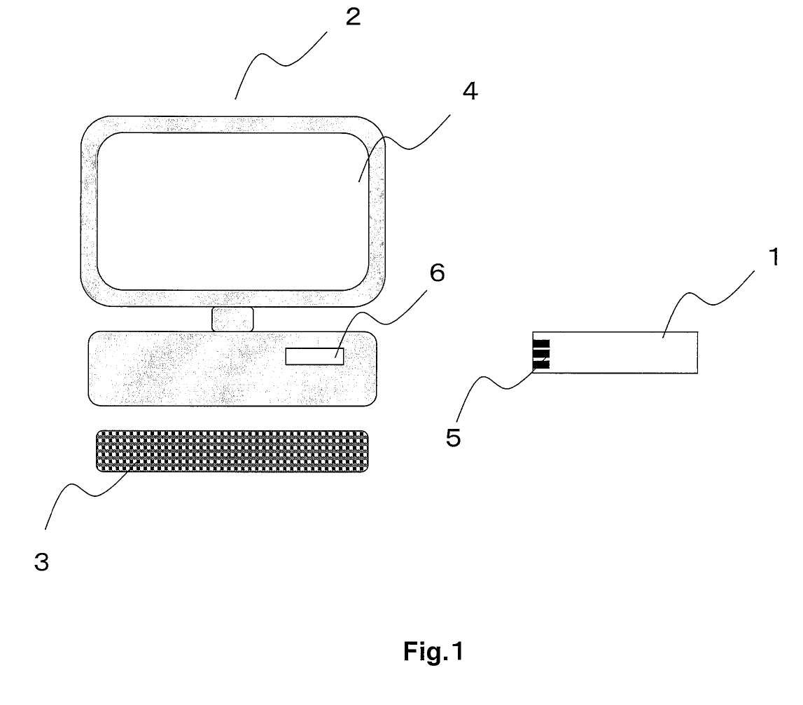

The apparatus for determining the health condition according to Embodiment 1 is shown in FIG. 1.

The healthcare management apparatus according to this Embodiment comprises a detection instrument 1 used in blood components analysis, an analyzer 2 for analyzing a signal in the detection instrument and converting into relative values, an input means 3 such as a keyboard or a mouse, and a display 4 such as a liquid crystal display.

An external connection terminal 5 is provided on the detection instrument 1, and inserted into a connection slot 6 of the analyzer 2. The signal detected in the detection instrument 1 is delivered to the analyzer 2.

As detection instrument 1, analytic microchip sensor with microchannels proposed in Patent document 5 may be used. For example, in the healthcare management apparatus for determining the risk of progression to lifestyle diseases by using a specific proteins levels in blood, it is preferable to select at least one of adiponectin, leptin, resistin and TNF-α, preferably all of them, as a detection target. Moreover, the levels of other blood components such as neutral fat, HDLc and blood glucose may be detected. Regarding the analytic microchip sensor used in these measurements, one microchip sensor may be used for each component, or may detect multiple components.

The analytical microchip sensor can be used for detecting other biological components in urine or salivary.

The detection instrument 1 is not essential to the system according to the present invention. For example, the present invention may adopt the configuration in which measured blood components data is input using an input means, or the data is obtained via wired or wireless communication lines.

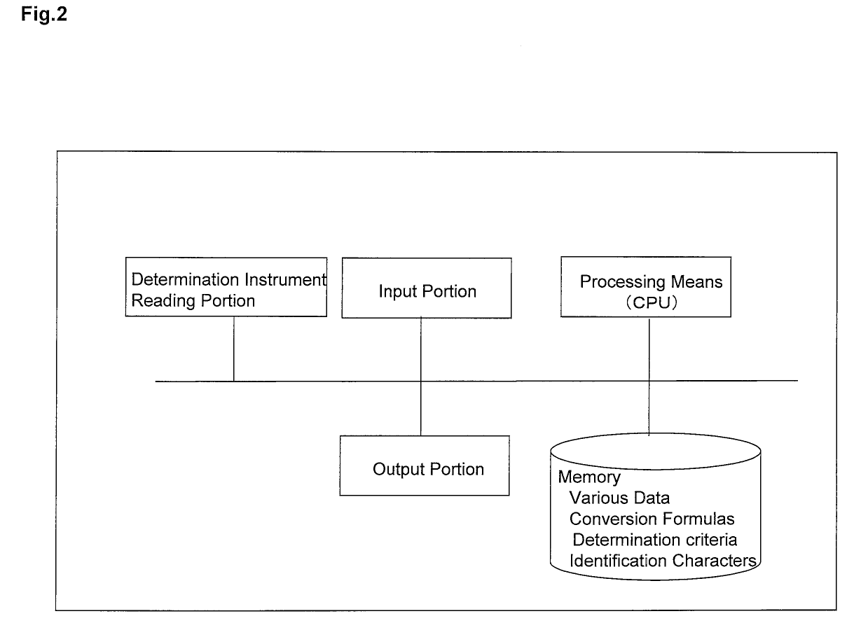

As shown in FIG. 2, the analyzer 2 comprises a processing means such as a central processing unit (CPU), a acquiring means having a memory such as hard disk drive or Flash SSD (Flash Solid State Drive), an input portion receiving an input of a input means and the like, an output portion outputting to a display and the like, and an detection instrument reading portion.

The memory stores various data such as physical data, blood components data and identification characters, conversion formula (such as pattern tables) used to calculate relative values, determination criteria, and a method for the health improvement that corresponds to the determination results. These may be stored in the same or different memories.

The above acquiring means may comprise a communication means to acquire various data via communication lines, instead of or together with the memory. As communication lines, wired or wireless communication may be used.

Identification characters of subjects are input from the input means 3, for example. The identification characters include, for example, each person's name, identification number or identification mark, age, sex, race and the like. Any one or more of these may be input in combination. Usually, it is convenient to contain a subject's name in the identification characters. Moreover, it is preferable to contain physical data that is not changed quickly in the daily life (for example, height of an adult person). Also, the identification characters may be acquired via a communication means.

The physical data and blood component data may be input from the input means. Or, as the physical data and blood component data, the data into which the analyzer 2 converts signal from detection instrument 1 connected to the analyzer 2 may be used. These data may be stored in a memory of the analyzer 2, or may be acquired via a communication means.

The display 4 displays the determination results and a health improvement method based on the results.

### (Health Condition Determination Method)

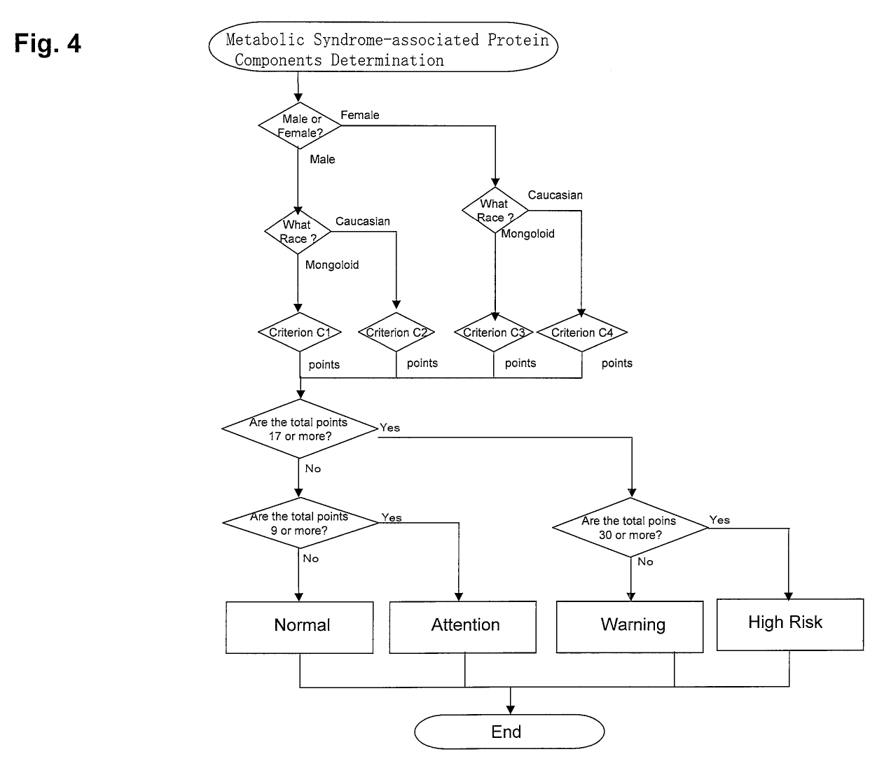

The health condition determination method according to this Embodiment is specifically explained using an example to determine the specific proteins in blood with reference to drawings. FIG. 4 is a diagram showing a flowchart of the health condition determination method (specific proteins in blood determination method) according to this Embodiment.

In this embodiment, the levels of adiponectin, leptin, resistin and TNF-α in blood are used as analysis data of blood components. And with the combination of these data, the risk of developing lifestyle diseases subjects is determined.

### (Pattern Table Deciding Step)

The Normal or High Risk ranges of the above four components would be different depending on sex, age, race and the like. Therefore, first, a suitable kind of pattern tables (data conversion formula) for a subject is decided.

The subject's identification characters stored in the memory are determined by a processing means of the analyzer 2, and then a pattern table according to a user's race (Mongoloid/Caucasian) and sex (male/female) is decided.

### (Detection Step)

Levels of the above four components in blood of the subject are detected using the detection instrument 1. In the detection, a conventional method may be used. For example, an analytic microchip sensor may be used as the detection instrument 1 to detect the levels of blood components using electrochemical method. And the electrochemical detection, an optical or electrical detection means may be also used.

Optical signals or an electrical signals sent from the detection instrument 1 are calculated at the analyzer 2 to work out each level of the above four components.

### (Relative Values Calculating Step)

The processing means of the analyzer 2 converts the levels of the above four components into the relative values using the pattern table (data conversion formula) decided in the above pattern table deciding step.

Table 1 shown below can be used as a pattern table classified by race and sex.

TABLE 1

| | Race/Sex | | | | | | | |
|---|---|---|---|---|---|---|---|---|
| | Mongoloid/Male (Criterion: C1) | | | | Mongoloid/Female (Criterion: C3) | | | |
| | Relative values | | | | | | | |
| | 0 | 5 | 8 | 10 | 0 | 5 | 8 | 10 |
| Adiponectin | >7.0 | 7.0-5.5 | 5.5-4.0 | <4.0 | >8.5 | 8.5-7.0 | 7.0-5.0 | <5.0 |
| (ug/mL) | | | | | | | | |
| Leptin | <3.5 | 3.5-6.5 | 6.5-9.5 | >9.5 | <3.0 | 3.0-6.0 | 6.0-12.0 | >12.0 |
| (ng/mL) | | | | | | | | |
| Resistin | <4.0 | 4.0-5.5 | 5.5-7.0 | >7.0 | <4.0 | 4.0-5.5 | 5.5-7.0 | >7.0 |
| (ng/mL) | | | | | | | | |
| TNF-α | <4.0 | 4.0-12.5 | 12.5-21.0 | >21.0 | <4.0 | 4.0-12.5 | 12.5-21.0 | >21.0 |
| (pg/mL) | | | | | | | | |
| | Race/Sex | | | | | | | |
| | Caucasian/Male (Criterion: C2) | | | | Caucasian/Female (Criterion: C4) | | | |
| | Relative values | | | | | | | |
| | 0 | 5 | 8 | 10 | 0 | 5 | 8 | 10 |
| Adiponectin | >7.0 | 7.0-5.5 | 5.5-4.0 | <4.0 | >8.5 | 8.5-7.0 | 7.0-5.0 | <5.0 |
| (ug/mL) | | | | | | | | |
| Leptin | <3.5 | 3.5-6.5 | 6.5-9.5 | >9.5 | <4.5 | 4.5-7.0 | 7.0-20 | >20 |
| (ng/mL) | | | | | | | | |
| Resistin | <4.0 | 4.0-5.5 | 5.5-7.0 | >7.0 | <4.0 | 4.0-5.5 | 5.5-7.0 | >7.0 |
| (ng/mL) | | | | | | | | |
| TNF-α | <4.0 | 4.0-12.5 | 12.5-21.0 | >21.0 | <4.0 | 4.0-12.5 | 12.5-21.0 | >21.0 |
| (pg/mL) | | | | | | | | |

### (Data Determination Step)

The analyzer 2 checks the resulting relative values with data determination criteria (detailed hereafter) stored in the memory to determine the respective data.

0 points: Normal (Stage 4)
5 points: Attention (Stage 3)
8 points: Warning (Stage 2)
10 points: High Risk (Stage 1)

### (Health Condition Determination Relative Values Calculation Step)

The processing means of the analyzer 2 sums up relative values the above four components and converts the sum into health assessment relative values using health assessment formula stored in the memory. As health condition formula, Table 2 shown below can be used.

TABLE 2

| Total points | Relative values |
|---|---|
| 0~8 | 0 |
| 9~16 | 3 |
| 17~29 | 7 |
| 30~40 | 10 |

### (Health Condition Determination Step)

In the processing means of the analyzer 2, the health condition determination relative values is checked up against the health condition determination criteria shown below to determine the risk of progression to lifestyle diseases of subjects.

### (Criteria of the Total Health Condition Determination)

0 points: Normal
3 points: Attention
7 points: Warning
10 points: High Risk

The data determination step and the relative values calculating step may be performed at the same time. Also, the health condition determination step and the health condition determination relative values calculating step may be performed at the same time.

### (Display Method of the Determination Results)

Next, a method for displaying the results of the above determination is explained.

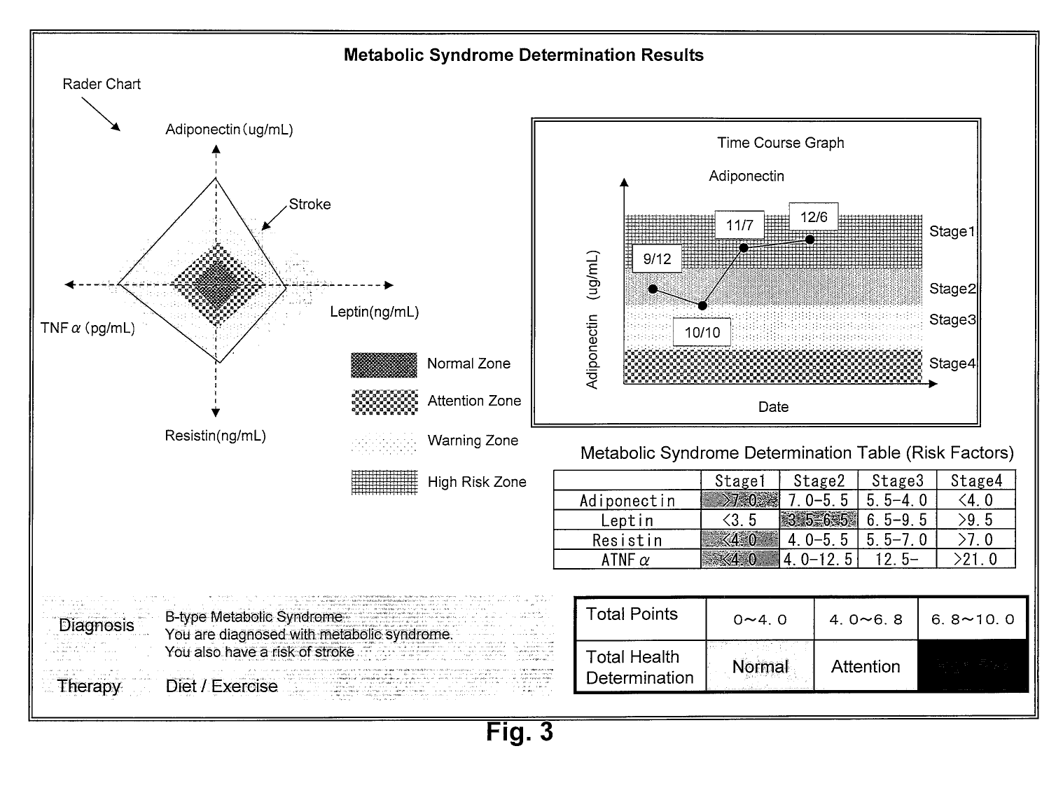

FIG. 3 shows an example of the display method according to the present invention. In this embodiment, it is exemplified that a display shows a radar chart showing respective levels of adiponectin, leptin, resistin and TNF-α along with its classification, a diagnostic determination table, a diagnostic results and a health improvement method.

The processing means compares the determination results with health improvement method data (therapeutic methods) stored in the memory, which correspond to the determination results, and thus shows an appreciate the health improvement method.

In this example of the display, the pattern table for “Mongoloid/Male” (Criterion C1) is used, and the determination results indicates adiponectin: Stage 1 (High Risk), leptin: Stage 2 (Warning), resistin: Stage 1 (High Risk) and TNF-α: Stage 1 (High Risk) (cf. the determination table and the radar chart).

The determination table shows the pattern table for “Mongoloid/Male” and the stage into which each determination of the above four components is classified.

On the radar chart color-coded on the basis of correspondence between level and stage (relative values), the level of the above adiponectin, leptin, resistin and TNF-α is plotted and then each point is connected by solid lines.

In each axis of the radar chart, the degree of risk is increasing from the center to the outward. Therefore, lower adiponectin level is positioned more outside. In the case of the other three components, higher level is positioned more outside. In the classification displayed together with the components levels, “Normal” (0 points: Stage 4) is shown inside, and “High Risk” (10 points: Stage 1) is shown outside.

In addition, it is preferable to color code the results. For example, “Normal” (0 points) is green, “Attention” (5 points) is yellow, “Warning” (8 points) is orange, and “High Risk” (10 points) is red. Thereby, it is clarified what zone (health, attention, warning or high risk) the quadrangle formed by connecting the respective component levels is mainly contained in. As a result, the health condition can be visually understood.

In this radar chart, it is preferable that indices having a strong degree of the antagonistic relationship and/or the correlation are arranged adjacently so that the health condition is easily understood.

For example, the relationship among these four components is explained below.

It is known that metabolic syndrome results from the reason that the adiponectin is decreased due to the production of TNF-α from fat cells. Therefore, there is an antagonistic relationship between TNF-α and adiponectin. That is, the more TNF-α, the less adiponectin (cf. Non-patent document 1). Therefore, when adiponectin and TNF-α are placed at the axes adjacent to each other, their relationship is easy to be understood.

The relationship between metabolic syndrome and the ratio of adiponectin and leptin is reported more often than adiponectin and leptin itself (cf. Non-patent document 4). Therefore, adiponectin and leptin are placed at the axes adjacent to each other. Thereby, the absolute value of the slope of the solid lines connecting the value of adiponectin and leptin corresponds to the ratio of adiponectin and leptin, which is useful for the determination of metabolic syndrome.

In view of the above, each component is positioned as follows so that adiponectin and TNF-α, and adiponectin and leptin are arranged adjacently to each other, respectively.

| | |
|---|---|
| Adiponectin: upward | Leptin: rightward |
| Resistin: downward | TNF-α: leftward |

Thereby, the results of the specific proteins in blood determination (diagnosis) and the health improvement method respond to the determination results (therapeutic method) are displayed.

In addition, when one or more past data are displayed together with the latest data, time course information can be obtained and thus the effects of therapy and prevention can be understood at a glance. In this case, it is preferable that the latest data is visually discriminated from the past data. For example, the quadrangle in the radar chart is formed by solid lines for the latest data, and by dotted lines for the past data.

In such a display method, when the quadrangle become smaller compared with the past data, improved health is visually indicated. In contrast, when the quadrangle become larger, it means undesirable.

It is known that the above four components is closely related to lifestyle diseases associated with metabolic syndrome. For example, it is reported that high leptin level and low adiponectin level result in the higher risk of a stroke (cf. Non-patent document 1). Therefore, when at least one of the component levels exceed the criteria value (for example, when the relative value of the above determination is 10 points: Stage 1), the prompt meaning the high risk of progression to lifestyle diseases (a warning message) is indicated to a user. This helps the user to figure out their symptoms at a glance.

In addition, for example, when the plots in the screen (input means) would be pointed by cursor, the plot of each component levels would be shown and a time course graph that shows a time course of the respective component levels may be displayed. This graph shows time on the horizontal and the component levels on the vertical axis. That is, it shows the respective component levels for each measurement date. Similarly to the radar chart, it is preferable that “normal”, “attention”, “warning” and “high risk” are color coded in the time course graph.

This kind of graph provides a trend of change, and thus facilitates to judge whether exercise, diet or a medicine is effective or not.

### Embodiment 2

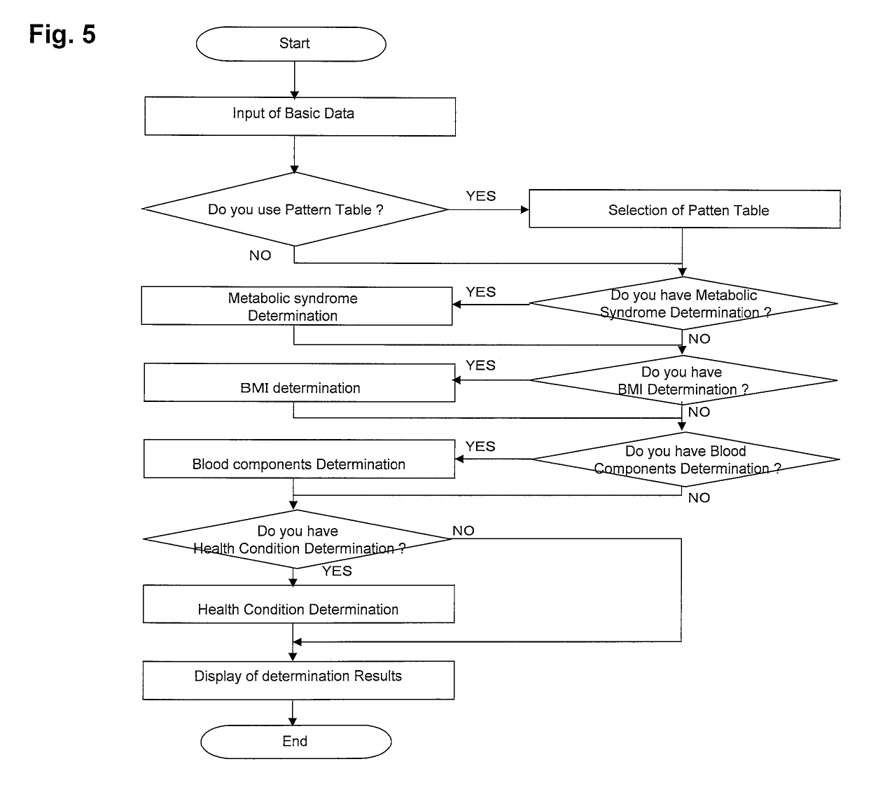

FIG. 5 is a diagram showing a flow chart of the health condition determining method according to Embodiment 2.

In this embodiment, an example is explained in which the following three determinations of health condition are performed, results of these determination are combined, and then a total health condition determination of subjects is performed. Since the configuration of the health condition determination apparatus is similar to Embodiment 1, its explanation is omitted.

(a) Metabolic syndrome determination
(b) BMI (Body Mass Index) determination
(c) Specific proteins in blood determination

In the metabolic syndrome determination, definitions disclosed by National Cholesterol Education Program (NCEP), International Diabetes Federation (IDF) and the Japan Society of Obesity (JASSO), etc. can be used, or other definitions can be also used.

In this embodiment, the definition of the International Diabetes Federation is used for the determination of metabolic syndrome. Therefore, the physical data and blood components data used in the metabolic syndrome determination are as follows.

Physical data: waist circumference, blood pressure

Blood components data: neutral fats level, HDLc level, blood glucose level

Also, the determination of specific proteins in blood is performed as described in Embodiment 1.

Therefore, the physical data and the blood component data used in this embodiment are shown below. For example, these data may be input by the input means and be stored in the memory.

Physical data: waist circumference, blood pressure, Body Mass Index (weight (kg)÷height (m)̂2)
Blood components data: neutral fat level, HDLc level, blood glucose level, adiponectin level, leptin level, resistin level, TNF-α level.

The memory stores data the conversion formula converting the physical data and the blood analysis data into relative values, the health assessment formula calculating health condition determination relative values from the relative values of the physical data and the blood analysis data, the total health assessment formula calculating health condition determination relative values from the health condition determination relative values, the data determination criteria, the health condition determination criteria, the total health condition determination criteria and the identifying characters, respectively.

### (Determination of Metabolic Syndrome)

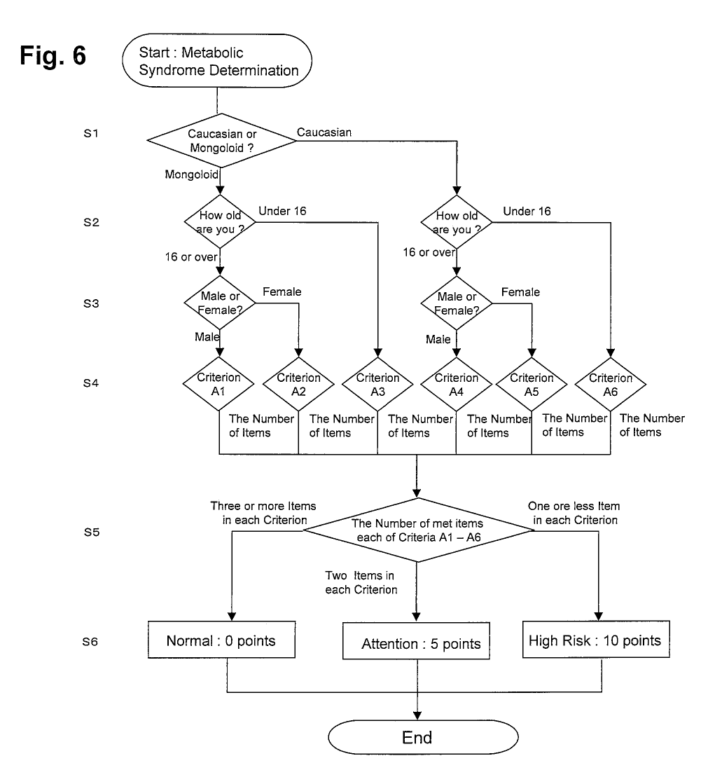

The algorithm to determine metabolic syndrome according to this embodiment is shown in FIG. 6.

### (Pattern Table Deciding Step)

First, the racial determination is made to divide the subjects into Mongoloid or Caucasian (S1).

Next, the age determination is made to divide the subjects into 16 or over or under 16 (S2).

In the case of 16 or over, the sex determination is performed (S3). In the case of under 16, the sex determination is not performed.

Based on S1 to S3, the subjects are divided into the following six groups. Then, the determination of metabolic syndrome is performed using the criterion (a pattern table) of each group (S4).

Mongoloid/Male/16 or over: Criterion A1
Mongoloid/Female/16 or over: Criterion A2

### Mongoloid/Male and Female/Under 16: Criterion A3

Caucasian/Male/16 or over: Criterion A4
Caucasian/Female/16 or over: Criterion A5

### Caucasian/Male and Female/Under 16: Criterion A6

The pattern table used in the determination of metabolic syndrome is shown in Table 3.

TABLE 3

| | Race | | | | | |
|---|---|---|---|---|---|---|
| | Mongoloid | | | Caucasian | | |
| | Sex | | | | | |
| | | | Male or | | | Male or |
| | Male | Female | Female | Male | Female | Female |
| Age | 16 or | 16 or | Under 16 | 16 or | 16 or | Under 16 |
| | over | over | | over | over | |
| Determination | A1 | A2 | A3 | A4 | A5 | A6 |
| criteria | | | | | | |
| Waist | ≧90 | ≧80 | ≧80 | ≧94 | ≧80 | ≧90 |
| circumference | | | | | | |
| (cm) | | | | | | |
| Blood pressure | ≧130/85 | ≧130/85 | ≧130/85 | ≧130/85 | ≧130/85 | ≧130/85 |
| (mmHg) | | | | | | |
| Neutral fat | ≧50 | ≧150 | ≧150 | ≧150 | ≧150 | ≧140 |
| (mg/dL) | | | | | | |
| HDLc | <40 | <50 | <40 | <40 | <50 | <40 |
| (mg/dL) | | | | | | |
| Blood glucose | ≧100 | ≧100 | ≧100 | ≧100 | ≧100 | ≧100 |
| level | | | | | | |
| (mg/dL) | | | | | | |

### (Relative Values Calculating/Data Determination Step)

According to the pattern table shown in Table 3, the individual physical data and blood components data are converted into the relative values. The waist circumference is divided into “match” (10 points) and “not match” (0 points). The items other than the waist circumference are divided into “match” (1 point) and “not match” (0 points).

### (Health Condition Determination Relative Values Calculating Step)

The individual physical data and the blood components data are summed up and the summed points are converted into the relative values for metabolic syndrome determination using the metabolic syndrome determination formula shown below (S5).

### (Metabolic Syndrome Determination Formula)

| | |
|---|---|
| The total points are 12 points or more: | 10 points |
| The total points are 2 to 4 points, or 11 points: | 5 points |
| The total points are 10 points, or 1 point or less: | 0 points |

### (Health Condition Determination Step)

The above relative values are assessed according to the metabolic syndrome determination criteria (health condition determination criteria) as shown below (S6).

### (Metabolic Syndrome Determination Criteria)

10 points: High Risk
5 points: Attention
0 points: Normal

### (BMI Determination)

BMI (Body Mass Index) is physical data represented by the following formula:

BMI=Body weight (kg)/Body height (m)̂2

### (Relative Values Calculating/Data Determination Step)

The BMI data is determined to divide into the four groups: High-degree obesity, Obesity, Normal and Slim. And each of the determination results is converted into relative values. The data determination criteria/data conversion formula of BMI is shown in Table 4.

TABLE 4

| Measurement values | Determination | Relative values |
|---|---|---|
| Over 30 | High-degree obesity | 10 points |
| 25 to 30 | Obesity | 8 points |
| 18.5 to 25 | Normal | 0 points |
| Under 18.5 | Slim | 3 points |

### (Specific Proteins in Blood Determination)

The specific proteins in blood determination are performed with similar method to Embodiment 1.

### (Total Health Condition Determination Relative Values Calculating Step)

The total health condition determination relative values are calculated by combining the relative values of the above three different determination results. In the total health assessment formula used for calculating the total health condition determination relative values, each determination result is obtained as shown below.

### (Total Health Assessment Formula)

Health condition determination relative values=(0.4×metabolic syndrome determination relative values)+(0.2×BMI determination relative values)+(0.4×specific proteins in blood determination relative values)

### (Total Health Condition Determination Step)

The resulting total health condition determination relative values are compared with the total health condition determination criteria shown below to determine the total health condition of the subjects.

### (Total Health Condition Determination Criteria)

0 to less than 4.0: Normal
4.0 to less than 6.8: Attention

### 6.8 to 10.0: High Risk

For example, if the metabolic syndrome determination is “High Risk”, BMI determination is “Obesity”, and the specific proteins in blood determination is “High Risk”, then the total health condition determination relative value is calculated as follows:

Total health condition determination relative values=0.4×10+0.2×8+0.4×8=8.8

Therefore, the total health condition is determined as “High Risk”.

Although, body weight is dependent on volume (cube of body height), since BMI indicates body weight divided by square of body height, a tall parson tends to be determined as “Obesity”. For this reason, another BMI determination criteria for a tall person (for example, 185 cm or more) may be used.

### Embodiment 3

In this embodiment, when any of the three health condition determinations are determined as “High Risk” in Embodiment 2, the total determination is defined as “High Risk”. The Step to calculate the health condition determination relative values and the steps before it are similar to Embodiment 2, and thus the explanation is omitted.

### (Metabolic Syndrome Determination)

The metabolic syndrome determination criteria is change as follows.

### (Metabolic Syndrome Determination Criteria)

| | |
|---|---|
| The total points are 12 points or more: | 100 points |
| The total points are 2 to 4 points, or 11 points: | 5 points |
| The total points are 10 points or 1 point or less: | 0 points |

### (BMI Determination)

The data determination criteria/data conversion formula is changed as follows.

### (BMI Data Determination Criteria)

| | |
|---|---|
| Over 30: | 100 points |
| 25 to 30: | 8 points |
| 18.5 to 25: | 0 points |
| Under 18.5: | 3 points |

### (Specific Proteins in Blood Determination)

The health condition determination criteria is changed as follows.

### (Metabolic Syndrome-Associated Protein Determination Criteria)

| | |
|---|---|
| 0~8: | 0 points |
| 9~16: | 3 points |
| 17~29: | 7 points |
| 30~40: | 100 points |

The total health assessment formula is similar to Embodiment 2. The total health condition determination criteria is as follows.

### (Total Health Condition Determination Criteria)

0 to less than 4.0: Normal
4.0 to less than 6.8: Attention
6.8 or more: High Risk

In the total health assessment according to this embodiment, when at least one of metabolic syndrome determination estimated as “High Risk”, BMI determination estimated as “high-degree obesity” and specific proteins in blood determination estimated as “High Risk” are applied, the total health condition determination relative value is defined as 20 points or more, regardless of the other determinations. This allows early improvement of health.

Further, the determination criteria may be changed as follows.

### (Metabolic Syndrome Determination Criteria)

| | |
|---|---|
| The total points are 12 points or more: | 250 points |
| The total points are 2 to 4 points or 11 points: | 5 points |
| The total points are 10 points, or 1 point or less: | 0 points |

### (BMI Data Determination Criteria)

| | |
|---|---|
| Over 30: | 5000 points |
| 25 to 30: | 8 points |
| 18.5 to 25: | 0 points |
| Under 18.5: | 3 points |

### (Specific Proteins in Blood Determination Criteria)

| | |
|---|---|
| 0~8 points: | 0 points |
| 9~16 points: | 3 points |
| 17~29 points: | 7 points |
| 30~40 points: | 25000 points |

In this case, when the above total health assessment formula is used, the hundreds digit of the total point indicates whether the metabolic syndrome determination is “High Risk” or not, the thousands digit indicates whether the BMI determination is “High-degree obesity” or not, and the ten thousands digit indicates whether the specific proteins in blood determination is “High Risk” or not. This provides an easy determination of the total health condition.

### Embodiment 4

In this embodiment, the method for displaying the determination results is explained. The determination method is performed according to Embodiments 2 and 3.

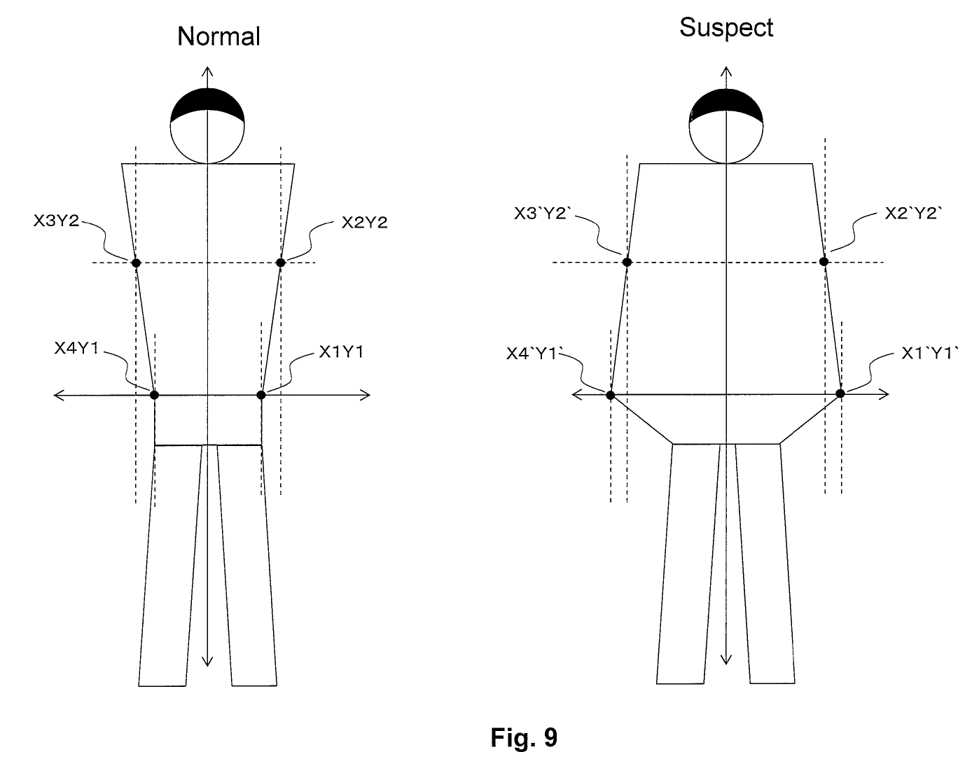

FIG. 9 shows the display method according to this embodiment. In FIG. 9, X1 is the level of adiponectin, X2 is the level of leptin, X3 is the level of resistin, and X4 is the level of TNF-α. Y1 and Y2 is a coefficient of obesity degree determined by Body Mass Index relative value.

The dash (′) attached to the BMI determination means the decision of obesity by BMI determination. When the dash (′) is not attached, it means normal in view of BMI determination.

In FIG. 9, the parameters is shown using a human-shaped model, which displays X1Y1 at the right edge of the waist, X2Y2 at the right edge of the chest, X3Y2 at the left edge of the chest, and X4Y1 at the left edge of the waist.

Such a display forms facilitates to visually understand whether of the risk of progression to lifestyle diseases is low (FIG. 9 (a)) or high (FIG. 9 (b)).

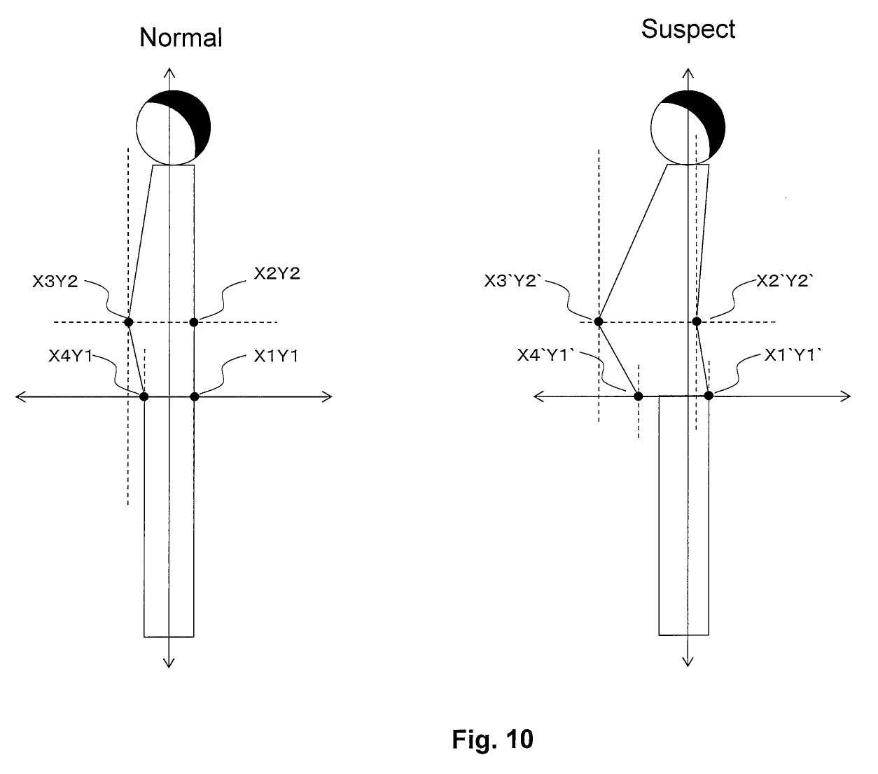

In addition, as shown in FIG. 10, the parameters may be shown using another human-shaped model, which displays X1Y1 at the back edge of the waist, X2Y2 at the back edge of the abdomen, X3Y2 at the front edge of the abdomen, and X4Y1 at the front edge of the waist.

As described above, according to the present invention, the health condition and the risk of progression to lifestyle diseases of subjects can conveniently determined. This can promote early health improvement of subjects and provides a noticeable effect in the prevention of lifestyle diseases. Therefore, the significance of the present invention is great.

What is claimed is:

1. A healthcare management apparatus comprising:
an acquiring means for acquiring physical data and blood components data of subjects, data conversion formula and data determination criteria; and
a processing means for converting the physical data and the blood components data into relative values by using the data conversion formula, and for determining health condition of the subjects by using the data determination criteria.

2. The healthcare management apparatus of claim 1, wherein:
the acquiring means further acquires health assessment formula and health condition determination criteria; and
the processing means combines two or more of the physical data, the blood components data and the relative values to calculate health condition determination relative values using the health assessment formula, and then checks the health condition determination relative values with the health condition determination criteria to determine a risk of progression to lifestyle diseases of subjects.

3. The healthcare management apparatus of claim 2, wherein:
the acquiring means further acquires total health assessment formula and total health condition determination criteria; and
the processing means combines two or more of the relative values and the health condition determination relative values to calculate total health condition determination relative values using the total health assessment formula, and then checks the total health condition determination relative values with the total health condition determination criteria to determine a total health condition of subjects.

4. The healthcare management apparatus of claim 1, wherein:
the acquiring means further acquires identification characters including information on sex, age and race of subjects.

5. The healthcare management apparatus of claim 4, wherein:
the data conversion formula is a pattern table that shows a relationship between measurement values and relative values.

6. The healthcare management apparatus of claim 5, wherein:
multiple kinds of the pattern tables are acquired according to sex, age and race; and
the processing means determines at least one of sex, age and race of subjects, and decides the pattern table to be used.

7. The healthcare management apparatus of claim 1, further comprising an input means.

8. The healthcare management apparatus of claim 1, further comprising a display for displaying determination results.

9. The healthcare management apparatus of claim 8, wherein:
the acquiring means further acquires health improvement method data; and
the processing means checks determination results with the health improvement method data, and lets the display show the determination results as well as a health improvement method corresponding to the determination results.

10. The healthcare management apparatus of claim 9, wherein:
the acquiring means further acquires time course information of determination results; and
the display further shows the time course information.

11. The healthcare management apparatus of claim 1, further comprising a detection instrument performing a blood components analysis
wherein,
the processing means calculates the blood components data from signals detected in the detection instrument.

12. The healthcare management apparatus of claim 1, wherein,
the acquiring means comprises a communication means for acquiring various information via communication lines.

13. A health condition determination method comprising:
a relative values calculating step for converting physical data and/or blood components data into relative values by using a data conversion formula; and
a data determination, step for determining whether the relative values meet data determination criteria or not.

14. The health condition determination method of claim 13 further comprising:
a health condition determination relative values calculating step for calculating relative values used in health condition determination by combining two or more of the relative values and using health assessment formula; and
a health condition determination step for determining a risk of progression to lifestyle diseases of subjects by checking the health condition determination relative values with health condition determination criteria.

15. A health condition determination method comprising;
a data determination step for determining whether physical data and/or blood components data of subjects meet data determination criteria or not;
a relative values conversion step for converting the physical data and the blood components data into relative values based on the data determination results;
a health condition determination relative values calculating step for calculating relative values used in health condition determination by combining two or more of the relative values and using health assessment formula; and
a health condition determination step for determining a risk of progression to lifestyle diseases of subjects by checking the health condition determination relative values with health condition determination criteria.

16. The health condition determination method of claim 14 further comprising:
a total health condition determination relative values calculating step for calculating relative values used in total health condition determination by combining two or more of the relative values and/or the health condition determination relative values and using total health assessment formula; and
a total health condition determination step for determining a total health condition of subjects by checking the total health condition determination relative values with total health condition determination criteria.

17. The health condition determination method of claim 15 further comprising:
a total health condition determination relative values calculating step for calculating relative values used in total health condition determination by combining two or more of the relative values and/or the health condition determination relative values and using total health assessment formula; and
a total health condition determination step for determining a total health condition of subjects by checking the total health condition determination relative values with total health condition determination criteria.

18. The health condition determination method of claim 14, wherein
the health condition determination relative values and/or the relative values include at least one selected from the group consisting of Body Mass Index relative values, metabolic syndrome determination relative values and specific proteins in blood determination relative values.

19. The health condition determination method of claim 15, wherein
the health condition determination relative values and/or the relative values include at least one selected from the group consisting of Body Mass Index relative values, metabolic syndrome determination relative values and specific proteins in blood determination relative values.

20. The health condition determination method of claim 18, wherein
blood components data used in the specific proteins in blood determination includes at least one analysis data of adiponectin, leptin, resistin and TNF-α.

21. The health condition determination method of claim 19, wherein
blood components data used in the specific proteins in blood determination includes at least one analysis data of adiponectin, leptin, resistin and TNF-α.

22. The health condition determination method of claim 13, wherein
a pattern table showing a relationship between measurement values and relative values is used as the data conversion formula.

23. The health condition determination method of claim 22 further comprising,
a pattern table deciding step for deciding the pattern table by determining at least one of sex, age and race of subjects.

24. A display method of health condition determination results comprising,
a display step for displaying determination results of the health condition determination method of claim 21 together with a graph showing relative values of the analysis data of adiponectin, leptin, resistin and TNF-α.

25. The display method of health condition determination results of claim 24,
wherein
the graph is arranged so that indices having a strong degree of the antagonistic relationship and/or correlation are placed adjacently to each other.

26. The display method of health condition determination results of claim 24, further comprising a step for displaying time course information of measurement values of the four components together with the graph.

27. The display method of health condition determination results of claim 24, further comprising a step for displaying a warning message when any determination result of the four components exceeds a criteria value.

28. The display method of health condition determination results of claim 24, further comprising a step for displaying the determination results and a health improvement method of subjects corresponding to the determination results.

29. The display method of health condition determination results of claim 24, further comprising a step for displaying X1Y1, X2Y2, X3Y2 and X4Y1, wherein:
X1 is the adiponectin level; X2 is the leptin level; X3 is the resistin level; X4 is the TNF-α level; and Y1 and Y2 are coefficients based on an obesity degree determined from Body Mass Index relative values.

30. The display method of health condition determination results of claim 29, further comprising a step for displaying X1Y1 at the right edge of the waist, X2Y2 at the right edge of the chest, X3Y2 at the left edge of the chest, and X4Y1 at the left edge of the waist in a human-shaped model.

31. The display method of health condition determination results of claim 29, further comprising a step for displaying X1Y1 at the back edge of the waist, X2Y2 at the back edge of the abdomen, X3Y2 at the front edge of the abdomen, and X4Y1 at the front edge of the waist in a human-shaped model.
